(19) United States
(12) Patent Application Publication
(10) Pub. No.: US 2005/0256462 A1
(43) Pub. Date: Nov. 17, 2005

(54) Nozzle

(76) Inventor: John R. Underwood, Laguna Nigel, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/845,791

(22) Filed: May 14, 2004

(51) Int. Cl. A61M 25/00

(52) U.S. Cl. 604/264; 604/22

(57) ABSTRACT

A tip nozzle for a surgical handpiece that may be used with existing tips. The nozzle seals the heated fluid injection port and restricts the size of the aspiration port. The nozzle is particularly useful dealing the irrigation/aspiration (“I/A”) portion of the surgical procedure.

### BACKGROUND OF THE INVENTION

This invention relates generally to the field of cataract surgery and more particularly to a handpiece tip for practicing the liquefracture technique of cataract removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted concurrently with the injection of the heated solution and the injection of a relatively cool solution, thereby quickly cooling and removing the heated solution. This technique is more fully described in U.S. Pat. No. 5,616,120 (Andrew, et al.), the entire content of which is incorporated herein by reference. The apparatus disclosed in the publication, however, heats the solution separately from the surgical handpiece. Temperature control of the heated solution can be difficult because the fluid tubes feeding the handpiece typically are up to two meters long, and the heated solution can cool considerably as it travels down the length of the tube.

U.S. Pat. No. 5,885,243 (Capetan, et al.) discloses a handpiece having a separate pumping mechanism and resistive heating element. Such a structure adds unnecessary complexity to the handpiece.

U.S. Pat. No. 6,579,270 B2 (Sussman, et al.) discloses a surgical handpiece and tip having two coaxial tubes or channels mounted within a body. The first tube is used for aspiration and is smaller in diameter than the second tube so as to create an annular passage between the first and second tube. The annular passage communicates with a pumping chamber formed between two electrodes. The pumping chamber works by boiling a small volume of the surgical fluid. As the fluid boils, it expands rapidly, thereby propelling the liquid downstream of the pumping chamber out of the annular passage. The distal end of the annular gap is sealed by a nozzle at the distal ends of the first and second tube and a plurality of orifices or ports may be formed in the nozzle. As the expanding gas is propelled down the annular gap, the gas/liquid stream is forced out of the distal orifice in a controlled and directed manner. However, aspiration and irrigation flow patterns different that those described in this patent are sometimes desired, such as during cortical clean up or posterior capsule washing or lavage.

Therefore, a need continues to exist for a simple surgical handpiece and tip that can heat internally the solution used to perform the liquefracture technique.

### BRIEF SUMMARY OF THE INVENTION

The present invention improves upon the prior art by providing a tip nozzle for a surgical handpiece that may be used with existing tips. The nozzle seals the heated fluid injection port and restricts the size of the aspiration port. The nozzle is particularly useful dealing the irrigation/aspiration (“I/A”) portion of the surgical procedure.

Accordingly, one objective of the present invention is to provide a tip nozzle for a surgical handpiece having at least two coaxial tubes.

Another objective of the present invention is to provide a tip nozzle for a handpiece having a pumping chamber.

Another objective of the present invention is to provide a tip nozzle for a surgical handpiece suitable for use during the I/A portion of a lens removal surgical procedure.

These and other advantages and objectives of the present invention will become apparent from the detailed description and claims that follow.

### BRIEF DESCRIPTION OF THE DRAWINGS

FIG. 1 is a partial cross-sectional view of a handpiece tip having the tip nozzle of the present invention inserted.

FIG. 2 is a partial cross-sectional view of the tip nozzle illustrated in FIG. 1.

### DETAILED DESCRIPTION OF THE INVENTION

Handpieces suitable for use with the present invention include the INFINITI® AQUALASE® surgical system which is commercially available from Alcon Laboratories, Inc., Fort Worth, Tex. This system uses a tip generally described in FIGS. 23 and 24 and column 7, lines 33-45 of U.S. Pat. No. 6,579,270 B2 (Sussman, et al.) and these portions of such patent are specifically incorporated herein by reference. As described in this patent, tip 900 may alteratively consist of outer tube 965 surrounding and coaxial with inner tube 967. Distal tip 902 of outer tube 965 is flared or belled so as to allow nozzle 905 to be inserted between outer tube 965 and inner tube 967. As best seen in FIG. 23, nozzle 905 contains fluid channel 907 that communicates with orifice 904. Nozzle 905 seals annular gap 969 between outer tube 965 and inner tube 967. Pressurized fluid flowing down annular gap 969 is forced into fluid channel 907 and out orifice 904.

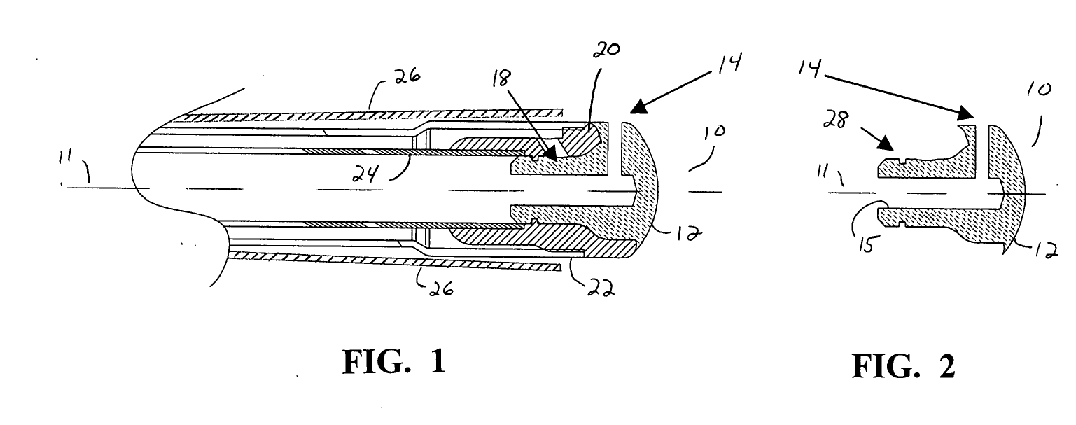

As best seen in FIGS. 1 and 2, tip nozzle 10 of the present invention generally includes rounded tip cap or body 12 having bore 15 in fluid communication with aspiration port 14. Nozzle made be of any suitable construction such as molded plastic or rubber. Body 12 is inserted into tip 20 and is held in place in tip 20 by a connector such as snap connector 28 so as to seal injection port 18 in handpiece tip 20. Aspiration port 14 is offset at an angle relative to centerline 11 of outer tube 22 and nozzle 10. Aspiration port 14 is generally smaller in diameter than bore 15 or inner aspiration tube 24. Such a construction allows for finer control of the tip and is particularly useful during the irrigation/aspiration (“I/A”) portion of a lens removal surgical procedure, such as cortical clean up. Coaxial irrigation sleeve 26 provides a source of irrigation fluid, if needed, as injection port 18 is plugged by nozzle 10.

This description is given for purposes of illustration and explanation. It will be apparent to those skilled in the relevant art that changes and modifications may be made to the invention described above without departing from its scope or spirit. For example, it will be recognized by those skilled in the art that the present invention may be combined with ultrasonic and/or rotating cutting tips to enhance performance.

1. (canceled)

2. A tip for a handpiece, comprising:
a) an inner tube having a bore mounted within an outer tube;
b) a annular gap formed between the inner tube and the outer tube; and
c) a tip nozzle sealing the annular gap from the bore at the distal ends of the inner tube and the outer tube, the tip nozzle being formed separately from the inner tube and the outer tube the tip nozzle having an aspiration port in fluid communication with the bore, the aspiration port being generally smaller in diameter than the bore.

3. The tip of claim 2 wherein the tip nozzle has a centerline, the aspiration port being offset at an angle relative to the centerline.

4. The tip of claim 2 wherein the tip nozzle has a connector suitable for holding the tip nozzle within the bore.
